(19) United States
(12) Patent Application Publication
(10) Pub. No.: US 2011/0027388 A1
(43) Pub. Date: Feb. 3, 2011

(54) Cobalt Hexammine as a Potential Therapeutic Against HIV and/or Ebola Virus

(75) Inventors: Eddie L. Chang, Silver Spring, MD (US); Lisa Hensley, Frederick, MD (US); Dzung C. Thach, Annandale, VA (US); Andy Knight, New Orleans, LA (US); Gene Olinger, Frederick, MD (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignees: The Government of the US, as represented by the Secretary of the Navy, Washington, DC (US); The Government of the United States, as represented by the Secretary of the Army

(21) Appl. No.: 12/847,342

(22) Filed: Jul. 30, 2010

Related U.S. Application Data
(60) Provisional application No. 61/230,287, filed on Jul. 31, 2009.
(60) Provisional application No. 61/261,018, filed on Nov. 13, 2009.

(51) Int. Cl. A61K 33/24; A61P 31/18; A61P 31/14; A61P 31/12; A61J 1/05; A61M 5/00

(52) U.S. Cl. 424/646; 604/403; 604/264; 604/272

(57) ABSTRACT

Hexaamminecobalt(III) chloride, also called Cohex, reduces the extent of viral infection, including difficult to treat infections caused by Ebola virus and HIV. Disclosed are methods for treating a viral infection, comprising administering to a patient a cobalt(III) hexammine compound in an amount effective to reduce an extent of a viral infection. Also disclosed are kits for delivery of a cobalt(III) hexammine compound by injection.

### CROSS-REFERENCE TO RELATED APPLICATIONS

This application claims the benefit of U.S. Provisional Application 61/230,287 filed on Jul. 31, 2009. This application also claims the benefit of U.S. Provisional Application 61/261,018 filed on Nov. 13, 2009. Each of these applications is incorporated by reference in its entirety.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Hexaamminecobalt(III) chloride, also called Cohex, is notable for its ability to “condense” dsDNA into toroidal-like superstructures under low salt conditions. The metal ion itself, Co(III), with its high positive charge density, is an ideal candidate for binding nucleotides with their high negative charge density. Although Co(III) is not stable by itself in aqueous solutions, it is stabilized by coordinating with donor atoms (usually N) that make strong contributions to the ligand field. These coordinating donors could either be monodentate ligands, e.g., NH₃, or polydentate chelators, such as cyclen, C₈H₂₀N₄. The Co(III)-chelator complexes (e.g., cobalt cyclen complexes) have been used for mechanistic studies of phosphodiester cleavage for both its efficient hydrolysis rates and kinetic inertness, whereby the kinetic inertness of Co(III) ions results in the continued binding of the complex to the hydrolyzed phosphate.

Due to the kinetic inertness of Co(III) ions, the Cohex complex sequesters the “inner-sphere” ammonia ligands from most exchange-reactions in solution; therefore, the usual interactions with solution molecules are by “outer-sphere” coordination via water bridges to the ammonia ligands and via the high charge-density of the Co(III) ion. These two characteristics play an important role in the strong attachment of Cohex to either DNA or RNA and in enabling Cohex to often substitute for hydrated Mg²⁺(aq) as a cofactor in nucleic acid biochemistry.

For example, Cohex complexation with 5S RNA—where Cohex was used in place of Mg²⁺(aq)—was found to provide no significant shifts in the λₘₐₓ of the absorption bands of Cohex, indicating that Cohex interaction with RNA was through outer-sphere complexation (and, of course, opposing charge attraction). It has also been reported that the number of binding sites on RNA was similar for Cohex and Mg²⁺(aq) and that the number was greater than expected for simple charge neutralization of the RNA backbone. These observations demonstrate that Cohex has a great propensity to bind to nucleotides at sites similar to Mg²⁺-binding sites and either inhibit or slow down the bio-functions of DNA and RNA.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

### BRIEF SUMMARY

Cohex can inhibit viral transcription/translation via interference with viral RNA. This interference can be either via general “blockade” of the nucleotide strands from transcription/translation or may be made more overt by attaching hybridizing oligonucleotide strands to the Cohex. It has been shown that Cohex does not hydrolyze nucleotides, but does show potent antiviral properties against the Sindbis virus and Adenovirus, which are positive single-stranded (ss) RNA, double-strand (ds) DNA, respectively, and furthermore can act as an antibiotic. See US Patent Application Publication Nos. 2008/0182835 and 2010/0004187, each of which is incorporated by reference in its entirety.

In one embodiment, a method for treating a viral infection comprises administering to a patient a hexaamminecobalt(III) compound (e.g., hexaamminecobalt(III) chloride) in an amount effective to reduce an extent of a viral infection.

In a further embodiment, a method for treating a viral infection comprises administering to a human patient a hexamminecobalt(III) compound in an amount effective to reduce an extent of an infection of the patient with Ebola virus or HIV.

In another embodiment, a kit for delivery of a hexamminecobalt(III) compound by injection comprises a hexamminecobalt(III) compound in a pharmaceutically acceptable carrier, and equipment for delivery thereof by injection, wherein the equipment comprises at least one of a container, injection tubing, or an injection needle.

### BRIEF DESCRIPTION OF THE DRAWINGS

FIG. 1 is an illustration of hexacoordinated Co(III), hexamminecobalt(III) (chloride counterions not shown), and magnesium(II) hexahydrate, Mg(H₂O)₆²⁺, both form octahedral coordination geometry with their respective ligands.

FIG. 2 is a double-Y semi-log plot is shown of the decrease in RT activity (left), as a measure of viral activity, or uninfected cell viability (right) for HIV-1 NL4-3 isolate. “% VC” means “% Virus Control” and “% CC” means “% Cell Control.”

FIG. 3 is a double-Y semi-log plot is shown of the decrease in RT activity (left), as a measure of viral activity, or uninfected cell viability (right) for HIV-1 Ba-L isolate. “% VC” means “% Virus Control” and “% CC” means “% Cell Control.”

FIG. 4 plots levels of GFP expression in cells infected with Zaire Ebola GFP, normalized against infected cells with no therapeutic (+/−control). Left plot: Relative GFP levels for A549 cells as a function of Cohex concentration, from 2.5 μM to 5 mM. Right plot: Relative GFP levels for HepG2 cells as a function of Cohex concentration

FIG. 5 plots of the levels of GFP expression in cells infected with Zaire Ebola GFP, normalized against infected cells with no therapeutic (+/−control). Left plot: Relative GFP levels for 293T cells as a function of Cohex concentration, from 2.5 μM to 5 mM. Right plot: Relative GFP levels for VeroE6 cells as a function of Cohex concentration.

FIG. 6 shows semi-log plots of the % viable (live) cells as a function of Cohex concentration. Left plot: A549 cells. Right plot: HepG2 cells.

FIG. 7 shows linear plots of the same data as FIG. 6, showing the region of greatest cytotoxic effect. Left plot: A549 cells. Right plot: HepG2 cells.

FIG. 8 shows linear plots of the % viable (live) cells as a function of Cohex concentration. Left plot: VeroE6 cells. Right plot: 293T cells.

FIG. 9 shows results for flow cytometric assay using PI as a marker for dead cells show almost no change between 0 to ˜1.2 mM Cohex.

FIG. 10 shows a curve fit of inhibition by Cohex. For purposes of fitting, the negative (−%) inhibitory % were turned into positive numbers; so 100%=100% inhibition. The IC50 for the fit was found to be 0.38 mM Cohex.

### DETAILED DESCRIPTION

Hexaamminecobalt(III) (Cohex; FIG. 1), in particular the chloride salt thereof, is notable for its ability to “condense” dsDNA into toroidal-like superstructures under low salt conditions. The metal ion itself, Co(III), with its high (+)charge-density, is an ideal candidate for binding nucleotides with their high (−)charge density. Although Co(III) is not stable by itself in aqueous solutions, it is stabilized by coordinating with donor atoms (usually N) that make strong contributions to the ligand field. These coordinating donors could either be monodentate ligands, e.g., NH₃, or polydentate chelators, such as cyclen, C₈H₂₀N₄. The Co(III)-chelator complexes (e.g., cobalt cyclen complexes) have been used for mechanistic studies of phosphodiester cleavage for both its efficient hydrolysis rates and kinetic inertness, whereby the kinetic inertness of Co(III) ions results in the continued binding of the complex to the hydrolyzed phosphate.

Due to the kinetic inertness of Co(III) ions, the Cohex complex sequesters the “inner-sphere” ammonia ligands from most exchange-reactions in solution; therefore, the usual interactions with solution molecules are by “outer-sphere” coordination via water bridges to the ammonia ligands and via the high charge-density of the Co(III) ion. These two characteristics play an important role in the strong attachment of Cohex to either DNA or RNA⁵ and in enabling Cohex to often substitute for hydrated Mg²⁺(aq) as a cofactor in nucleic acid biochemistry. For example, Cohex complexation with 5S RNA—where Cohex was used in place of Mg²⁺(aq)—was examined and found to provide no significant shifts in the λₘₐₓ of the absorption bands of Cohex, indicating that Cohex interaction with RNA was through outer-sphere complexation (and, of course, opposing charge attraction). It has also been reported that the number of binding sites on RNA was similar for Cohex and Mg²⁺(aq) and that the number was greater than expected for simple charge neutralization of the RNA backbone. These observations demonstrate that Cohex has a great propensity to bind to nucleotides at sites similar to Mg²⁺-binding sites and either inhibit or slow down the bio-functions of DNA and RNA.

Cohex may function as a new type of broad-spectrum antiviral compound. For example, Cohex can be effective in significantly enhancing cell viability and in depressing viral expression for Sindbis infected BHK cells, with similar significant effects of Cohex against adenovirus in A549. See US Patent Application Publication No. 2008/0182835. These observations point to the potential broad-spectrum nature of Cohex against viruses.

As disclosed herein, Cohex demonstrates antiviral properties against two additional viruses. Ebola virus is a negative-strand, filamentous, enveloped microorganism that belongs to the filoviridae family of viruses. Cohex can decrease the viral expression levels in a dose-dependent manner, in a variety of cells infected with the Ebola virus. Cohex also demonstrates antiviral properties against human immunodeficiency virus (HIV). HIV is a member of the genus lentivirus and belongs to the Retroviridae family. It has a single-strand (−)RNA genome, which is transcribed into a complementary DNA (cDNA) inside the host cell by an RNA-dependent DNA polymerase. The sense cDNA serves as a template for DNA-dependent DNA polymerase to make an antisense DNA copy, which forms a double-stranded viral DNA (dsDNA). The dsDNA is then transported into the cell nucleus where it gets integrated into the host cell's genome. Virus replication is initiated when the integrated DNA provirus is transcribed into mRNA.

### DEFINITIONS

As used herein, the term “reduce an extent of the viral infection” with regard to a patient means that the ability of viruses to multiply within a patient is at least partially reduced.

As used herein, a “patient” can be a human or other mammal.

### Antiviral Uses of Cohex

It is contemplated that Cohex could be used to treat a viral infection in a patient. In one embodiment, an effective amount of Cohex is administered to a patient suspected or known to have a viral infection. Optionally, a method of treatment includes identifying a patient who is or may be in need of such treatment. The patient can be a human or other mammal, including without limitation a primate, dog, cat, cow, pig, or horse.

In an embodiment, Cohex is administered to a patient known or suspected of being infected by a virus. In a further embodiment, Cohex is administered prior to exposure of the patient to a virus. In another embodiment, Cohex is administered subsequent to exposure of the patent to a virus.

The Cohex may be administered by any of various means including orally or nasally, or by suppository, or by injection including intravenous, intramuscular, or intraperitoneal injection, or combinations of any of these.

In an embodiment, equipment for injection of Cohex in a pharmaceutically acceptable comprises at least one of a container for the compound (such as a tube, bottle, or bag), injection tubing, or an injection needle.

The quantity of Cohex effective to treat an infection can be ascertained by one of ordinary skill in the art. Exemplary amounts of Cohex include 0.5, 1, 2, 4, 8, 10, 12, 14, 16, 18, or 20 mg/kg, or more.

Viral infections that can be treated include, but are not limited to, those associated with human immunodeficiency virus (HIV), human T cell leukemia virus (HTLV), Papillomavirus (e.g., human papilloma virus), Polyomavirus (e.g., SV40, BK virus, DAR virus), orthopoxvirus (e.g., variola major virus (smallpox virus)), EBV, herpes simplex virus (HSV), hepatitis virus, Rhabdovirus (e.g., Ebola virus), alphavirus (e.g., Sindbis virus), adenovirus, and/or cytomegalovirus (CMV). In preferred embodiments, the viral infection is by HIV or Ebola virus.

### Preparation of Co(III) Hexammine

While Cohex is available commercially, its synthesis is fairly straight forward, using air to oxidize Co(II) to Co(III):

CoCl₂+4NH₄Cl+20NH₃+O₂→4[Co(NH₃)₆]Cl₃+2H₂O

9.6 g of CoCl₂.6H₂O (0.06 mol) and 6.4 g of NH₄Cl (0.12 mol) were added to 40 ml of water in a 250 ml Erlenmeyer flask with a side arm and shaken until most of the salts are dissolved. Then 1 g of fresh activated decolorizing charcoal and 20 ml concentrated ammonia were added. Next the flask was connected to the aspirator or vacuum line and air drawn through the mixture until the red solution becomes yellowish brown (usually 2-3 hours). The air inlet tube if preferably of fairly large bore (˜10 mm) to prevent clogging with the precipitated Co(NH₃)₆³⁺ salt.

The crystals and charcoal were filtered on a Buchner funnel and then a solution of 6 ml of concentrated HCl in 75 ml of water was added. The mixture was heated on a hot plate to effect complete solution and filtered while hot. The hexamminecobalt (III) chloride was crystallized by cooling to 0° C. and by slowly adding 15 ml of concentrated HCl. The crystals were filtered, washed with 60% and then with 95% ethanol, and dried at 80-100° C.

### Cohex Activity Against HIV

There are two known strains of HIV: HIV-1 and HIV-2, of which HIV-1 is the more virulent virus and is the major cause of HIV infections. The first clinically useful drugs developed for HIV-1 were the nucleoside reverse transcriptase (RT) inhibitors. AZT, or 3-azido-3-deoxythymidine, is a synthetic pyrimidine analog of thymidine was actually initially developed as an anticancer drug before it became known as a popular anti-HIV compound. The active form of AZT is its phosphorylated triphosphate (TP) form, which is a competitive inhibitor of RT because AZT-TP binds to the HIV-1 RT better than to the natural substrate deoxythymidine triphosphate (dTTP).

Cohex was tested in a standard PBMC cell-based microtiter anti-HIV assay against one CXCR4-tropic HIV-1 isolate and one CCR5-tropic HIV-1 isolate. For this study peripheral blood mononuclear cells (PBMCs) were pre-treated with the compound for two hours prior to infection.

Cohex was stored at 4° C. as a powder and solubilized for tests. The solubilized stock was stored at −20° C. until the day of the assay. Stocks were thawed at room temperature on each day of assay setup and were used to generate working drug dilutions used in the assays. Working dilutions were made fresh for each experiment and were not stored for re-use in subsequent experiments performed on different days. Cohex was evaluated using a 3 mM (3,000 μM) high-test concentration with 8 additional serial half-log dilutions in the PBMC assays.

### PBMC Assay

Freshly prepared PBMCs were centrifuged and suspended in RPMI 1640 with 15% FBS, L-glutamine, penicillin, streptomycin, non-essential amino acids (MEM/NEAA; Hyclone; catalog #SH30238.01), and 20 U/ml recombinant human IL-2. PBMCs were maintained in this medium at a concentration of 1-2×10⁶ cells/ml, with twice-weekly medium changes until they were used in the assay protocol. Monocyte-derived-macrophages were depleted from the culture as the result of adherence to the tissue culture flask.

For the standard PBMC assay, the cells were plated in the interior wells of a 96 well round bottom microplate at 50 μL/well (5×10⁴ cells/well) in a standard format developed by the Infectious Disease Research department of Southern Research Institute. Each plate contains virus control wells (cells plus virus) and experimental wells (drug plus cells plus virus). Test drug dilutions were prepared at a 2× concentration in microtiter tubes and 100 μL of each concentration was placed in appropriate wells using the standard format. 50 μL of a predetermined dilution of virus stock was placed in each test well (final MOI ˜0.1). Separate plates were prepared identically without virus for drug cytotoxicity studies using an MTS assay system (described below; cytotoxicity plates also include compound control wells containing drug plus media without cells to control for colored compounds that affect the MTS assay). The PBMC cultures were maintained for seven days following infection at 37° C., 5% CO₂. After this period, cell-free supernatant samples were collected for analysis of reverse transcriptase activity and compound cytotoxicity was measured by addition of MTS to the separate cytotoxicity plates for determination of cell viability. Wells were also examined microscopically and any abnormalities were noted.

### Reverse Transcriptase Activity Assay

A microtiter plate-based reverse transcriptase (RT) reaction was utilized (detailed in Buckheit et al., AIDS Research and Human Retroviruses 7:295-302, 1991). Tritiated thymidine triphosphate (3H-TTP, 80 Ci/mmol, NEN) was received in 1:1 dH₂O:Ethanol at 1 mCi/ml. Poly rA:oligo dT template:primer (Pharmacia) was prepared as a stock solution by combining 150 poly rA (20 mg/ml) with 0.5 ml oligo dT (20 units/ml) and 5.35 ml sterile dH₂O followed by aliquoting (1.0 ml) and storage at −20° C. The RT reaction buffer was prepared fresh on a daily basis and consisted of 125 μl 1.0 M EGTA, 125 μl dH2O, 125 μl 20% Triton X100, 50 μl 1.0 M Tris (pH 7.4), 50 μl 1.0 M DTT, and 40 μl 1.0 M MgCl₂. The final reaction mixture was prepared by combining 1 part 3H-TTP, 4 parts dH₂O, 2.5 parts poly rA:oligo dT stock and 2.5 parts reaction buffer. Ten microliters of this reaction mixture was placed in a round bottom microtiter plate and 15 μl of virus-containing supernatant was added and mixed. The plate was incubated at 37° C. for 60 minutes. Following incubation, the reaction volume was spotted onto DE81 filter-mats (Wallac), washed 5 times for 5 minutes each in a 5% sodium phosphate buffer or 2×SSC (Life Technologies), 2 times for 1 minute each in distilled water, 2 times for 1 minute each in 70% ethanol, and then dried. Incorporated radioactivity (counts per minute, CPM) was quantified using standard liquid scintillation techniques.

### MTS Staining for PBMC Viability to Measure Cytotoxicity

At assay termination, the uninfected assay plates were stained with the soluble tetrazolium-based dye MTS (CellTiter 96 Reagent, Promega) to determine cell viability and quantify compound toxicity. MTS is metabolized by the mitochondria enzymes of metabolically active cells to yield a soluble formazan product, allowing the rapid quantitative analysis of cell viability and compound cytotoxicity. This reagent is a stable, single solution that does not require preparation before use. At termination of the assay, 20-25 μL of MTS reagent is added per well and the microtiter plates are then incubated for 4-6 hrs at 37° C., 5% CO₂ to assess cell viability. Adhesive plate sealers were used in place of the lids, the sealed plate was inverted several times to mix the soluble formazan product and the plate was read spectrophotometrically at 490/650 nm with a Molecular Devices SPECTRAmax plate reader.

### Assay Results

The PBMC data were normalized by dividing by either the average control, infected, untreated value for the infection measurements (% Viral Control) or by the control, uninfected, untreated value for the cytotoxicity measurements (% Cell Control). The normalized values were then analyzed for IC50 (50% inhibition of virus replication), CC50 (50% cytotoxicity), and therapeutic index values (TI=CC/IC; also referred to as Antiviral Index or AI).

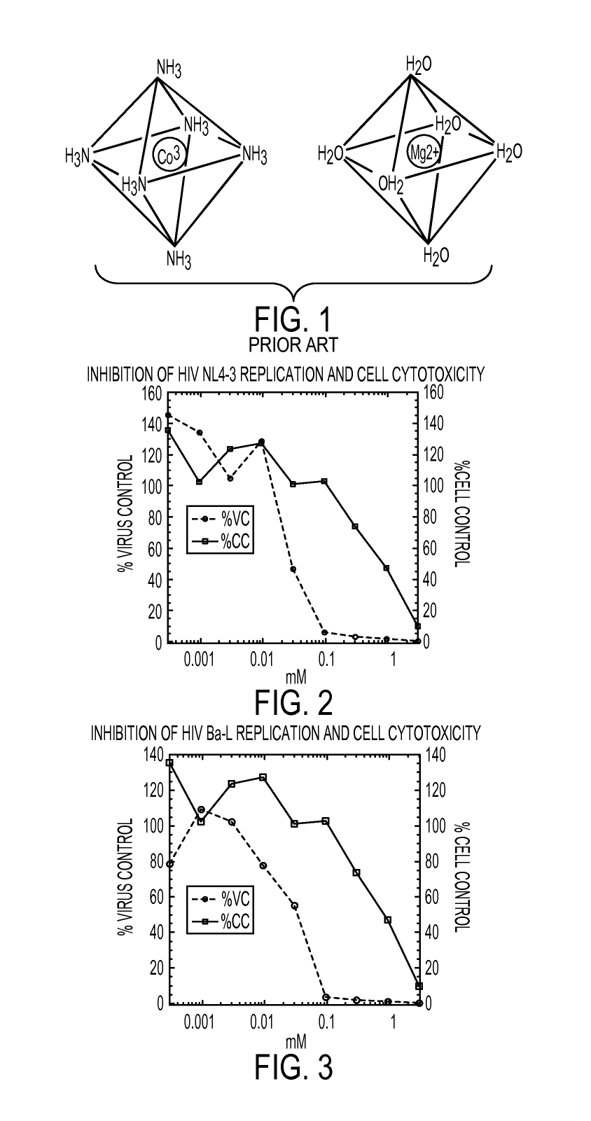

Cohex was tested for antiviral efficacy against one CXCR4-tropic HIV-1 isolate and one CCR5-tropic HIV-1 isolate in PBMCs. For this study PBMCs were pre-treated with the compound for two hours prior to infection. FIG. 2 illustrates the decrease in RT activity (left), as a measure of viral activity, or uninfected cell viability (right) for HIV-1 NL4-3 isolate. FIG. 3 illustrates of the decrease in RT activity (left), as a measure of viral activity, or uninfected cell viability (right) for HIV-1 Ba-L isolate. In these Figures, “% VC” means “% Virus Control” and “% CC” means “% Cell Control.” The results of the testing are summarized in Table 1.

Cohex displayed definite antiviral activity against the virus isolates evaluated in this study, with an average IC50 value of 31.2 μM. There did not appear to be any difference in the activity of the compound based on co-receptor tropism, as the compound had approximately equal activity against both virus isolates tested. Cytotoxicity was observed with the compound at concentrations above 100 μM (TC50=833 μM), resulting in an average Therapeutic Index value of 26.7. These results can be summarized with IC50, CC50, and TI values given in Table 1.

TABLE 1
Summary of Cohex Activity Against HIV-1 in PBMCs

| | | | | Therapeutic |
|---|---|---|---|---|
| Compound | HIV-1 Isolate | IC₅₀ | CC₅₀ | Index |
| Cohex | Ba-L | 33.8 μM | 833 μM | 24.7 |
| | NL4-3 | 28.6 μM | | 29.1 |

The results show that Cohex displays very similar activity against HIV as against other types of viruses, attesting to the very broad-spectrum nature of the compound. The antiviral activity is not as high as specific antiviral drugs, like AZT, but there are situations where the use of Cohex can be an advantage.

### Cohex Activity Against Ebola Virus

Ebola was first discovered simultaneously in 1976 in Sudan and in the Democratic Republic of the Congo (formerly Zaire). While its origins are still not firmly established, Ebola likely came from the rain forests of Africa. The primary reservoir is likely not nonhuman primates, but rather that the virus is zoonotic, transmitted to humans from ongoing life cycles in animals or arthropods.

Ebola viruses belong to the filoviridae family and has five known strains (subtypes): Bundibugyo, Côte d'Ivoire, Sudan, Zaïre, and Reston. The Bundibugyo, Sudan, and Zaïre strains have caused outbreaks of Ebola hemorrhagic fever among humans in Africa, killing up to 90% of those infected. Of the Ebola viruses, the Zaire strain is the most virulent and the Reston strain is the least virulent.

The Ebola virus is transmitted via contact with bodily fluids of infected persons and can take from two days to three weeks for symptoms to appear. Disease symptoms start with fever, muscle aches and a cough before progressing to severe vomiting, diarrhea and rashes, along with kidney and liver problems. Death generally occurs as the result of either one or a combination of dehydration and/or massive bleeding from leaky blood vessels, kidney, and liver failure. The World Health Organization has documented 1,850 cases of Ebola (mostly in sub-Saharan Africa) since its discovery; only 600 (32 percent) of the victims survived. (32 percent) of the victims survived.

As with all viruses of the order Mononegavirales, filoviruses, such as Ebola, contain a single-stranded, negative-sense RNA molecule as their genome. The genomes of filoviruses are quite large at approximately 19,000 bases in length and contain seven sequentially arranged genes. Filovirus proteins can be subdivided into two categories, those that form the ribonucleoprotein (RNP) complex and those that are associated with the envelope. The proteins associated with the nucleocapsid are involved in the transcription and replication of the genome, whereas the envelope-associated proteins primarily have a role either in assembly of the virion or in receptor binding and virus entry.

There is no known cure for Ebola disease. Existing antiviral drugs do not work well against this virus and the best doctors can do is attempt to maintain the patient's body fluids and electrolytes levels under intensive care; while bleeding problems may require transfusions of platelets and/or fresh blood.

### Activity of Cohex Against Ebola Virus in Cell Culture

For EC50 assays, cells were plated onto 96-well plates and incubated at 37° C. for 24 hours before adding compound followed by cell infection with Zaire Ebola GFP virus, a virus strain that contains a GFP gene. The infected cells were allowed to grow for an additional 48 hours before reading on a Molecular Devices spectrofluorometer (X=485 nm, M=515 nm). Controls were done for +virus/−compound and −virus/−compound. The −virus/+compound controls were part of the CC50 tests. Dosage of Cohex ranged from 2.5 μM to 5 mM and were done in triplicates. Error bars for the figures are for standard error (SE) of the mean.

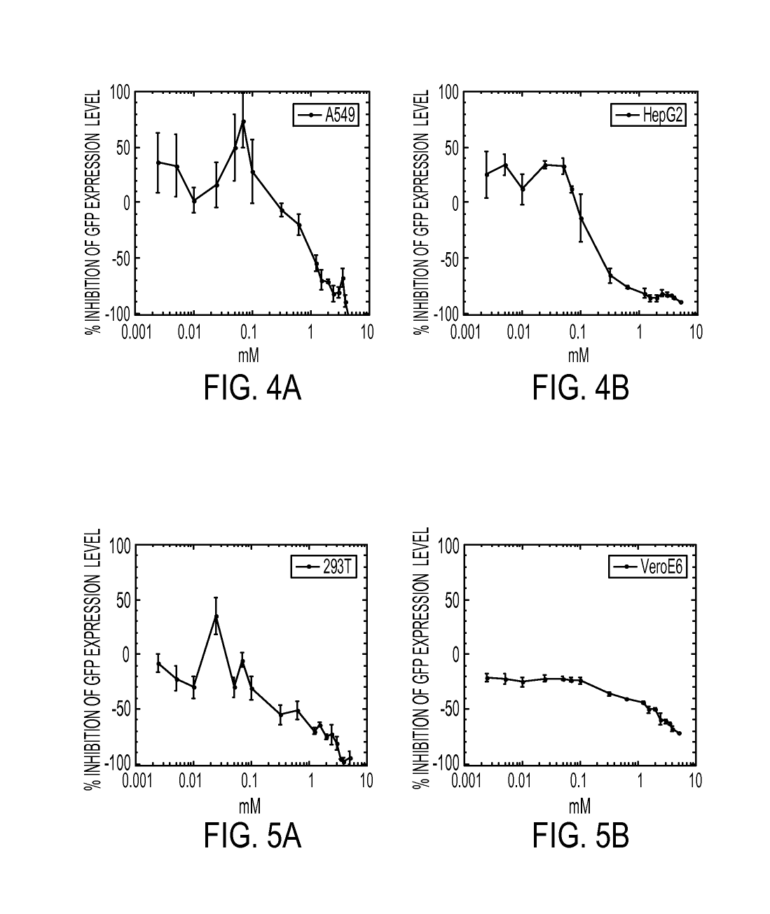

The results for A549 cells and HepG2 cells are shown in the left and right panels of FIG. 4, respectively. It is seen that there appears to be a general flat response from 2.5 μM until around 0.1 mM Cohex, at which point, GFP expression drops until there is nearly 100% suppression (−100%) of viral expression at concentrations above 1 mM Cohex.

The results for 293T and VeroE6 cells are shown in the left and right panels of FIG. 5, respectively. For 293T cells, there is a monotonic decrease in GFP expression with increasing Cohex, even starting as low as 2.5 μM Cohex. For VeroE6 cells, there is also a decrease in GFP expression with increasing Cohex, but the slope of the decrease is much less pronounced than for the other cells. There is another difference in the cells of FIG. 4 from FIG. 5. The values for concentrations below 0.1 mM in FIG. 1 fluctuate between 0 and +50 enhancement of GFP with large error bars, whereas the values in FIG. 2, for the same region of concentration, all show (except for 1 point) negative GFP enhancement (i.e., in the suppression of expression region). Thus, the behavior of Cohex for the different cell types exhibit differential amounts of viral expression decrease, but they all show decreasing levels of GFP fluorescence with increasing Cohex concentrations, especially above 0.1 mM.

In order to check whether the decreasing GFP levels were simply due to decreasing numbers of viable cells, in vitro cytotoxicity studies were performed for the same cell lines. That is, the same concentration ranges as used above were used in a CellTiter-Glo Luminescent Cell Viability Assay by Promega. This assay is based on quantitation of the ATP present in cells, which signals the presence of metabolically active cells, that is, a decrease in luminescence correlates with a decrease in the number of viable cells. The cells were plated out on 96-well plates, as above, and incubated at 37° C. for 24 hours before adding compound. The treated cells were then allowed to grow for an additional 48 hours before reading on the BMG Lumistar set on the ATP protocol.

In addition to the luminescence assay, a flow cytometry assay was performed using propidium iodide as a “dead” stain for A549 cells. The flow cytometry assay protocol for A549 cell line is similar to protocols known in the art, and is as follows. The cells were grown until confluent and reseeded at 100,000 cells/well in 1 ml in 24-well plates. The monolayers were allowed to form overnight at 37° C. under 5% CO₂. The Cohex dilution series was added to appropriate wells and the plate incubated for 48 hours at 37° C. under 5% CO₂. The cells were then washed, pelleted, resuspended in buffer, and transferred to BD falcon tubes for flow analysis. A BD FACSort cytometer and BD CellQuest software was used to quantify cell viability. Prior to flow analysis, 10 μL of propidium iodide (PI) at 0.05 mg/ml was added to each tube to stain dead cells. Analysis was performed on 1×10⁴ events/well.

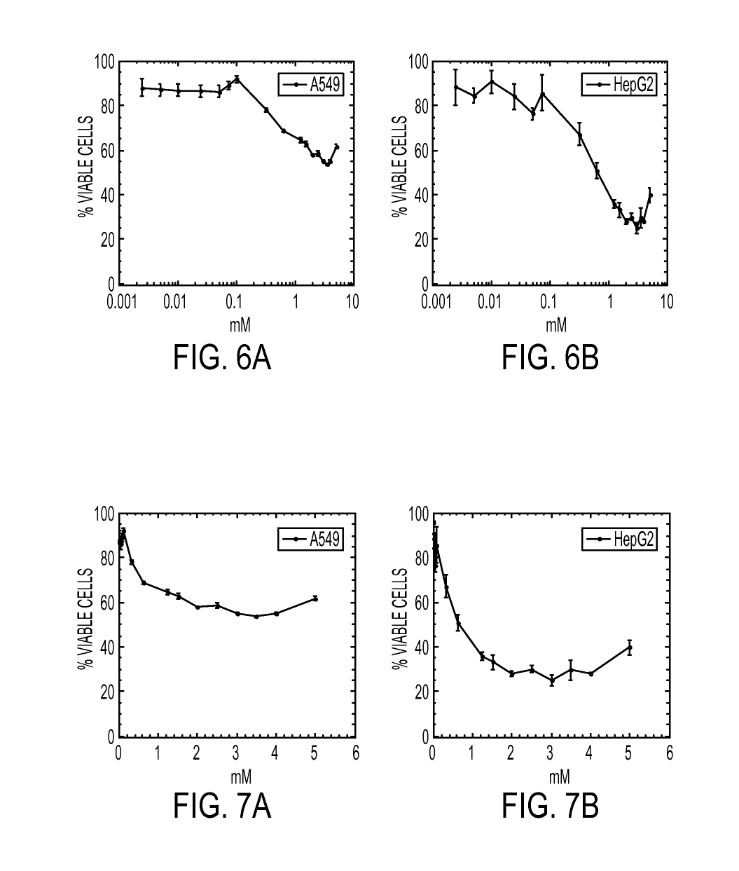

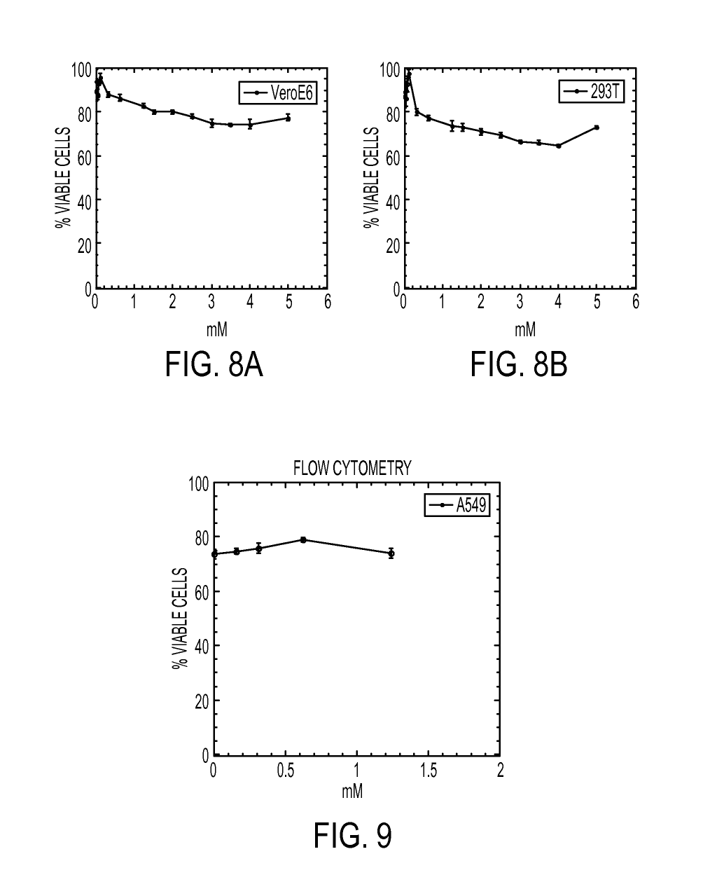

FIG. 6 shows the result of the cytotoxicity assay for A549 and HepG2 cells plotted on semi-log scale. There appears to be no toxic effect until about 0.1 mM, after which there is a decreasing % of viable cells. To better show the region from 2.5 μM to 0.1 mM, FIG. 7 provides linear-scale plots to emphasize the concentration region that does affect cytotoxicity.

Both 293T and VerE6 cells lines show much less cytotoxic susceptibility to Cohex, leveling off between 70 to 80% viability, even at 5 mM Cohex. There is a variety of reactions to Cohex by different cell lines, but none of the cells were 100% killed, whereas suppression of GFP expression tends to bottom out close to −100% (except for VeroE6).

It is further notable that, in addition to variability between cell lines, different markers can also differ in their assessment of viability. As an example, the results of a flow cytometry measurement using propidium iodide (PI) as a marker for dead cells shown in FIG. 9. it can be seen that PI appears to measure a cell property (cell permeability) that is much less affected by Cohex than the luminescence study (ATP levels).

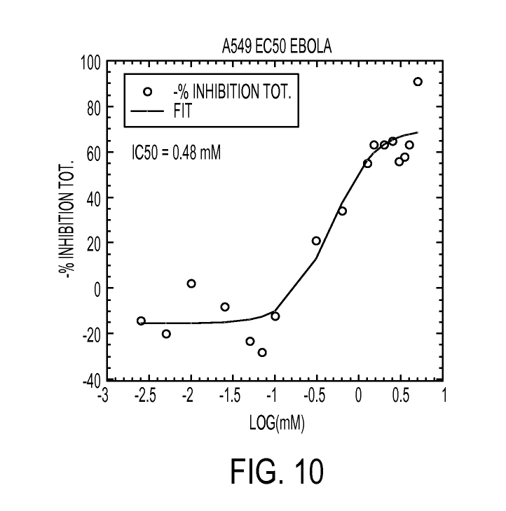

The IC50 for Cohex for the different cell lines can be estimated from FIGS. 1 and 2. By using a log concentration scale, the data can be fitted to the classic sigmoidal shape using a non-linear least-squares fitting program, seen in FIG. 10. The IC50 for the fit was found to be 0.38 mM Cohex.

The results with various cell types are shown in Table 2.

TABLE 2
Summary of Cohex IC50 for Various Cell Types

| | A549 | HepG2 | VeroE6 | 293T |
|---|---|---|---|---|
| IC50 (mM) | 0.48 | 0.24 | 1.66 | 1.28 |

### Cohex Animal Study Against Ebola

An efficacy study was conducted in mice to test whether Cohex would have a therapeutic affect against Ebola virus exposure. Initially, to determine whether the mice would tolerate the Cohex, they received intraperitoneal (IP) injections of Cohex once a day for 10 days at levels of 0.5, 1, 2, 4, and 8 mg/kg in this study. The mice tolerated the compound very well, with no adverse reactions reported.

To examine the efficacy of Cohex, mice were treated by IP injection with either phosphate buffered saline (PBS) or Cohex in PBS one hour before virus exposure, and further treated once a day for 9 more days. In comparing the results of the mice treated with PBS versus those treated with 8 mg/kg of Cohex, it was found to be statistically very likely (p=0.01 in a chi-squared test) that the 8 mg/kg treatment improved survival rates over the PBS treatment in mice infected with Ebola virus.

The general advantages of a broad-spectrum drug, such as Cohex, are its low-cost, stability, and, of course, ability to attack multiple microorganisms. When there is no treatment available, as in the case of Ebola virus, Cohex could be the only source of treatment. For viruses, such as HIV, where drugs with very high TI already exist, Cohex can be used in a combination drug therapy regime. There are several advantages to doing this: (1) as a broad-spectrum compound, Cohex can fight against opportunistic infections by other microorganisms; (2) Cohex may have a synergistic effect on existing anti-HIV drugs; (3) Cohex can significantly decrease the cost of anti-HIV treatment; (4) Cohex can slow the development of viral drug-resistance by presenting a very different mechanism that must be overcome.

All numbers expressing quantities of ingredients, constituents, reaction conditions, and so forth used in the specification are to be understood as being modified in all instances by the term “about.” Notwithstanding that the numerical ranges and parameters set forth, the broad scope of the subject matter presented herein are approximations, the numerical values set forth are indicated as precisely as possible. Any numerical value, however, may inherently contain certain errors resulting, for example, from their respective measurement techniques, as evidenced by standard deviations associated therewith.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed in accordance with 35 U.S.C. §112, ¶6 unless the term “means” is expressly used in association therewith.

1. A method for treating a viral infection, comprising administering to a patient a cobalt(III) hexammine compound in an amount effective to reduce an extent of a viral infection.

2. The method of claim 1, wherein the cobalt(III) hexammine compound is hexamminecobalt(III) chloride.

3. The method of claim 1, wherein the viral infection is by HIV or Ebola.

4. The method of claim 1, wherein the cobalt(III) hexammine compound is administered prior to the viral infection.

5. The method of claim 1, wherein the cobalt(III) hexammine compound is administered subsequent to the viral infection.

6. The method of claim 1 wherein said administering is performed both prior to said viral infection and after said viral infection.

7. The method of claim 1, further comprising identifying whether said patient is in need of antiviral treatment.

8. The method of claim 1, wherein the administering is by injection.

9. A method for treating a viral infection, comprising administering to a human patient hexamminecobalt(III) chloride in an amount effective to reduce an extent of an infection of the patient with Ebola virus or HIV.

10. The method of claim 9, wherein the infection is with the Ebola virus.

11. The method of claim 9, wherein the administering is by injection.

12. A kit for delivery of a cobalt(III) hexammine compound by injection, comprising:
a cobalt(III) hexammine compound in a pharmaceutically acceptable carrier, and equipment for delivery thereof by injection,
wherein the equipment comprises at least one of a container, injection tubing, or an injection needle.

13. The kit of claim 12, wherein the equipment comprises the container as well as (1) the injection tubing, (2) the injection needle, or (3) both the injection tubing and the injection needle.
